(19) United States
(12) Patent Application Publication
(10) Pub. No.: US 2008/0131535 A1
(43) Pub. Date: Jun. 5, 2008

(54) SKIN PROTECTION COMPOSITION

(75) Inventors: Yuuzou Tsuchida, Tokyo (JP); Kotarou Tsuchida, Tokyo (JP); Kenjirou Tsuchida, Tokyo (JP); Kunitomo Watanabe, Gifu-shi (JP); Yoshiko Nakamura, Sagamihara-shi (JP); Atsuo Iwasawa, Yokohama-shi (JP)

Correspondence Address: SUGHRUE MION, PLLC, 2100 PENNSYLVANIA AVENUE, N.W., SUITE 800, WASHINGTON, DC, 20037, US

(73) Assignee: Hououdou Co., Ltd., Tokyo (JP)

(21) Appl. No.: 12/026,383

(22) Filed: Feb. 5, 2008

Related U.S. Application Data
(62) Division of application No. 11/246,194, filed on Oct. 11, 2005.
Application No. 11/246,194 is a continuation of application No. PCT/JP03/04635, filed on Apr. 11, 2003.

(51) Int. Cl. A61K 36/899

(52) U.S. Cl. 424/750

(57) ABSTRACT

The present invention provides a method comprising suppressing the growth of a microorganism selected from the group consisting of anthrax bacteria and a virus selected from the group consisting of herpes simplex virus and influenza virus by providing an object containing or impregnated with a composition comprising Sasa extract, and bringing the microorganism into contact with the composition.

### TECHNICAL FIELD

The present invention relates to a skin protection composition, and more particularly to a skin protection composition that can effectively suppress intrusion of contagious bacteria and viruses from wounds and from mucosa such as those of the eyes, nose, throat, ears, anus and vagina into the body and also can suppress growth of the bacteria and viruses. The present invention also relates to an antiseptic. The present invention further relates to a filter apparatus that can effectively prevent intrusion into the body and growth therein of contagious bacteria and viruses.

### BACKGROUND ART

Contagious bacteria such as Pseudomonas aeruginosa, Bacillus subtilis, Bacillus anthracis, Escherichia coli, Staphylococcus aureus, and viruses such as the influenza virus float freely in the air, can intrude into the body from the mucosa and wound regions of humans and animals, and can cause severe infections in humans and animals. A variety of antibiotics are used for this kind of microbial infection. However, various kinds of antibiotic resistant bacteria such as methicillin-resistant staphylococcus aureus (MRSA), against which conventional antibiotics are not effective, have appeared as a result of the overuse of antibiotics. This results in patients, doctors and nurses in hospitals becoming infected with resistant bacteria, sometimes even leading to death.

Meanwhile, a mail containing anthrax was sent recently to politicians and mass media-related personalities in the U.S., and many of the unprotected people who opened the mail were infected with anthrax leading to mortalities, and causing widespread fear in society. Then, the desirability of having a suitable protective means against this kind of biological weapon not only for those preparing for public work, but also for general citizens quickly became apparent.

### DISCLOSURE OF THE INVENTION

Consequently, an object of the present invention is to provide a skin protection composition that can effectively suppress intrusion into the body and growth therein of contagious bacteria such as Pseudomonas aeruginosa, Bacillus subtilis, Bacillus anthracis, Escherichia coli, Staphylococcus aureus, MRSA, Gas bacillus, Tetanus bacillus, and Botulinus bacillus, and viruses such as herpes virus, influenza virus, and coronavirus from skin, wounds and from mucosa such as those of the eyes, nose, throat, ears, anus and vagina.

Another object of the present invention is to provide a skin protection composition that can effectively prevent so-called hospital infections by resistant bacteria.

Another object of the present invention is to provide a preventive and/or therapeutic composition for bacterial and virus infections including hospital infections.

A further object of the present invention is to provide an antiseptic.

A further object of the present invention is to provide a filter apparatus that can effectively suppress invasion and growth of infectious bacteria and viruses.

The present invention provides the following skin protection compositions, preventive and/or therapeutic compositions for bacterial and virus infections, antiseptics, and filter apparatuses.

1. A skin protection composition comprising Sasa extract as an active component.
2. A bacterial infection preventive and/or therapeutic composition comprising Sasa extract as an active component.
3. A viral infection preventive and/or therapeutic composition comprising Sasa extract as an active component.
4. A hospital infection preventive and/or therapeutic composition comprising Sasa extract as an active component.
5. The composition according to any one of the above 1 to 4 further comprising an organic acid.
6. The composition according to any one of the above 1 to 5 that is in the form of an oral composition.
7. The composition according to 6 above that is in the form of a confection such as a gummi, jelly, troche, candy, chewing gum, jam, sherbet, cream, drop, sponge cake, cookie, chocolate, or rice cracker, breads, noodles, beverages, tablets, pills, mouthwash, gargle, toothpaste, skin adhesive film, or spray agent for treating throat inflammation.
8. The composition according to any one of the above 1 to 5 that is in the form of a protective cloth.
9. The composition according to 8 above that is in the form of gauze, mask, eye patch, menstrual band, menstrual napkin, medical dressing, toilet paper, gauze for treating hemorrhoids, toilet seat sheet, shoe insert, ear plug, glove, hat, white gown, sheet, futon cover, pillow case, curtain, wall paper, carpet, and surgical suture thread.
10. The composition according to 8 above that is in the form of gauze.
11. A mask comprising the gauze according to 10 above.
12. An antiseptic agent comprising Sasa extract as an active component.
13. The antiseptic agent according to 12 above wherein the active component further comprises an organic acid.
14. A filter apparatus comprising Sasa extract as an active component.
15. The filter apparatus according to 14 above wherein the active component further comprises an organic acid.
16. The filter apparatus according to 14 or 15 above in the form of a filter used in a region that air passes through in a fan, air conditioning, air inlet, air outlet, screen door, air cleaner, or electric vacuum cleaner.
17. An air cleaner comprising the filter apparatus according to any one of the above 14 to 16.

### BEST MODE FOR CARRYING OUT THE INVENTION

The present invention will be explained in detail below.

### Sasa Extract

The Sasa (Sasa albo-marginata) used in the present invention, as a raw material for the Sasa extract is not restricted to any specific one and any plant belonging to the genus Sasa may be used herein. Specific examples thereof include those specified below: Kumai Sasa; Sasa albo-marginata Makino et Shibata (Kuma Sasa); ground bamboo; Okuyama Sasa; Ezo-Miyama Sasa; Sasa Paniculata Makino et Shibata; Yahiko Sasa; Oba Sasa; Miyama Sasa; Sendai Sasa; Yukawa Sasa; Aboi Sasa; and Onuka Sasa. Among these, specific examples of commercially available ones include Kumai Sasa and Kuma Sasa (Chugoku Sasa and Hida Sasa). For instance, preferably used herein are extracts derived from, for instance, Kumai Sasa and/or Kuma Sasa collected in, for instance, TESHIO Mountains in Hokkaido, Japan during the term extending from July to October. The extract used in the present invention is preferably obtained by normal pressure or pressurized extraction from fresh or dried leaves, preferably dried leaves, in 100 to 180° C. water.

The extraction method is not particularly limited, but may be, for example, the method described in Japanese Patent No. 3212278 (Japanese Unexamined Patent Application Publication No. H11-196818). Concretely, an extract is extracted by processing 5 to 30 minutes at 100 to 180° C. using a pressurized hot water extractor, and separating the extract in question from the moist solid parts (water content percentage 40 to 70%) using a water content separator. After using a saturated water steam heat processor to process the moist solid part at 100 to 200° C. for 5 to 60 minutes, the extract is again extracted by processing 5 to 30 minutes at 100 to 180° C. using a pressurized hot water extractor, and the first and second extracts are combined and used. An extract obtained by extracting from Sasa dried leaf in 60 to 100° C. water for about 30 minutes to 12 hours may also be used.

“AHSS” manufactured by Chloroland Moshiri Co., Ltd. is an example of a commercial product comprising 50% solid weight Sasa extract.

The Sasa extract obtained in this way comprises a sulfur component, and converted to sulfur, the content is approximately 4 to 10 mg per gram of solid weight Sasa extract, normally, 6 to 9 mg. The primary component of this sulfur constituent appears to be amino acid containing sulfur.

The composition of the present invention per 100 g solid extract preferably contains 4 to 500 mg of sulfur component derived from Sasa extract converted to sulfur; more preferably 8 to 250 g; and most preferably, 16 to 150 mg.

Moreover, Sasa extract contains tannin, and that content is about 5 to 15% by mass in relation to the solid content of the Sasa extract.

The composition of the present invention preferably contains 0.05 to 7.5% Sasa tannin by mass in dry component concentration, and more preferably 0.1 to 6% by mass.

The composition of the present invention may have only Sasa extract as the active component, and by combining with a suitable amount of organic acids, the therapeutic and/or preventive effects can be further improved. Malic acid, citric acid, lactic acid, oxalic acid, malonic acid, succinic acid, fumaric acid, acetic acid, benzoic acid, phenylacetic acid, salicylic acid, and phenols may be cited as this kind of organic acid.

The amount of organic acid used is preferably 0.01 to 5% by mass in the composition of the present invention; more preferably 0.02 to 3% by mass, and most preferably 0.05 to 1.5% by mass.

Skin protection composition, bacterial and viral infection preventive and/or therapeutic composition

The skin protection composition, bacterial and viral infection preventive and/or therapeutic composition of the present invention is a substance that effectively suppresses intrusion into the body of contagious bacteria and viruses from skin, from wounds and from mucous membranes such as those of the eyes, nose, throat, ears, anus and vagina, and effectively prevents and/or treats bacterial infections including hospital infections, and infections from such viruses as influenza virus, herpes virus, and coronavirus.

Skin protective cloth and oral compositions of the present invention may be cited as forms of the skin protection composition comprising Sasa extract as the active component, as well as of compositions to prevent and/or treat bacterial and viral infections.

Skin protective cloth comprises an air permeable support impregnated or sprayed with Sasa extract, the air permeable support including, for example, natural fibers such as silk, cotton, hemp or wool, synthetic fibers such as polyurethane, vinylon, polypropylene, nylon, polyester, or acrylic, semi-synthetic fibers, or fibers themselves, or threads, woven cloth, knitted cloth, non-woven cloth, or paper which is a mixture of two or more kinds of these. As a matter of convenience, in addition to cloth, “protective cloth” in the present specification shall include the fibers themselves, thread, paper or the like.

In addition to protective cloth that directly contacts mucosa and skin and is classified as a hygienic product such as gauze, masks, eye patches, menstrual bands, menstrual napkins, medical dressings, toilet paper, gauze for treating hemorrhoids, toilet seat sheets, shoe inserts, ear plugs, and plasters, examples of concrete forms include clothing such as white gowns, clothing items such as gloves, hats, socks, and Japanese socks, bedding such as sheets, futon covers, pillow cases, and bed articles, room interior accessories and materials such as curtains, wall paper and carpets, medical materials such as surgical suture thread, articles that directly or indirectly touch the skin, and cooking utensils such as chopping blocks.

Oral compositions comprising Sasa extract of the present invention include, for example, confections such as gummis, jellies, troches, candies, chewing gums, jams, sherbets, creams, drops, sponge cakes, cookies, chocolates, or rice crackers, breads, noodles, beverages, tablets, pills, mouthwash, gargle, toothpaste, skin adhesive film, or spray agents for treating throat inflammation.

To include Sasa extract in the skin protective cloth, the protective cloth is immersed in an aqueous solution of 2 to 20% Sasa extract by mass, or an aqueous solution of 2 to 20% Sasa extract by mass is sprayed on the protective cloth and dried. For a suitable amount of impregnated or sprayed Sasa extract, it is preferable to contain 2 to 20% Sasa extract by mass in the solid content, more preferably 6 to 15% by mass, and most preferably 8 to 12% by mass. If less than 2% by mass, the targeted effect will be insufficiently manifested; and if exceeding 20% by mass, the skin or mucosa may experience a burning sensation, and with little further improvement in effect there will be no economic advantage.

Per 100 g of protection composition, the skin protection composition of the present invention preferably contains 8 to 200 mg of sulfur component derived from Sasa extract converted to sulfur, more preferably 24 to 150 mg, and most preferably 32 to 120 mg.

To include Sasa extract in oral compositions such as, for example, in a confection such as a gummi, jelly, troche, candy, chewing gum, jam, sherbet, cream, drop, sponge cake, cookie, chocolate, or rice cracker, breads, noodles, beverages, tablets, pills (for example, “Sasatan”), mouthwash, gargle, toothpaste, of skin adhesive film, at every stage of manufacturing the oral composition, it is preferable to add 2 to 20% Sasa extract solid content by mass to the raw materials of the oral composition, more preferably 6 to 15% by mass, and most preferably 8 to 12% by mass. If less than 2% by mass, the targeted effect will be insufficiently manifested; and if exceeding 200% by mass, the skin or mucosa may experience a burning sensation, and with little further improvement in effect there will be no economic advantage.

When including, or after including the Sasa extract in the protective cloth or oral composition, it is preferable to heat process at a temperature of 70 to 90° C., more preferably, of 75 to 85° C., for 30 minutes to 3 hours, more preferably, for 1 to 2 hours. Heat processing dramatically improves the skin protection effect.

Suitable and customary components may be compounded in the oral composition of the present invention depending on the dosage form. For example, excipients such as fructose, lactose, sucrose, starch syrup, dextrin, and cyclodextrin, binders such as gum Arabic, sodium carboxymethyl cellulose, crystalline cellulose, and gum base, disintegrating agents such as starch, lubricants such as magnesium stearate, and sucrose fatty acid esters, and fresheners such as fragrance, chlorophyll, spearmint, mint, and menthol may be cited.

When using the skin protection composition of the present invention in the form of a protective cloth, Sasa extract is incorporated in the protective cloth (gauze, or the like) at the location that contacts the mucosa or skin region, and this protective cloth may be replaced about 1 to 3 times a day. When using in the form of surgical suture thread, intrusion of bacteria from the sutured wound area is suppressed, infection of the wound area is suppressed, and the healing of the surgical region is promoted.

If the skin protection composition of the present invention is used in the form of a protective cloth in order to prevent hospital infection at clinics or the like, the infection prevention effect of the skin protection composition of the present invention will continue for a long period of time. When that effect is reduced by laundering, the protective cloth may be suitably replaced or re-treated to impregnate Sasa extract again.

If the skin protection composition of the present invention is used in the form of an oral composition, the amount ingested is not particularly limited, but about 2 to 15 mg as Sasa extract solid content may be ingested, for example 1 to 5 times daily, normally 1 to 3 times. The amount and frequency of ingestion may be suitably increased or decreased to match objectives.

The skin protection composition of the present invention contains 2 to 20% Sasa extract solid content by mass, and indicates a marked antibacterial effect on resistant bacteria for which conventional antibiotics are not effective.

In addition, when using the skin protection composition of the present invention in the form of an oral composition, contagious bacterial and viral infections can be prevented and the growth thereof can be suppressed in an extremely simple manner by taking the composition orally, if suitable and necessary. In addition, gradual release forms of the composition such as chewing gum and candy have the advantage of manifesting that effect over a prolonged period of time.

Skin protection compositions of the present invention can be used in the form of ointments, creams, hair gels, gels, lotions, oils, soaps, and shampoos. Contagious bacterial and viral infections can be prevented and the growth thereof can be suppressed in an extremely simple manner by coating the skin with ointment, cream, hair gel, gel, lotion or oil (for example, jojoba oil, grape seed oil, and oryza oil) containing 2 to 20% Sasa extract solid content by mass.

### Antiseptic

The present invention also provides an antiseptic containing Sasa extract as the active component. The form when used as an antiseptic is not particularly limited. Food, beverages, condiments, cosmetics (including lotions and oils), sprays for treatment of wounds, and sprays for treatment of throat inflammations or the like may be cited as antiseptic compositions. It is preferable that 2 to 20% Sasa extract solid content by mass is contained in these target substances, more preferably 6 to 15% by mass, and most preferably 8 to 12% by mass. If less than 2% by mass, the targeted antiseptic effect will be insufficiently manifested; and if exceeding 20% by mass, there will be little further improvement in effect, and no economic advantage. In addition, if adding to foods, an undesirable change in the flavor of the food may occur.

### Filter Apparatus

The present invention also provides an air filter apparatus containing Sasa extract as the active component. Concrete forms include filters used in a region that air passes through in a fan, air conditioning, car air conditioning, air inlet, air outlet, screen door, air cleaner, electric vacuum cleaner, or the like. The filter material is not particularly limited, and includes natural fibers such as silk, cotton, wool or hemp, synthetic fibers such as polyurethane, polypropylene, nylon, polyester, acrylic, or vinylon, semi-synthetic fibers, glass fibers, or woven cloth, knitted cloth, non-woven cloth, or paper which is a mixture of two or more kinds of these. These filters may be impregnated with aqueous dilute solutions of Sasa extract by immersion, spraying or the like, and then dried. For a suitable amount of Sasa extract in the filter, it is preferable to include 2 to 20% Sasa extract solid content by mass, more preferably 4 to 15% by mass, and most preferably 6 to 8% by mass. If less than 2% by mass, the targeted disinfectant, bacterial elimination, and growth prevention effects will be insufficiently manifested; and if exceeding 20% by mass, there will be little further improvement in effect and no economic advantage. When passing through this filter, bacteria and viruses present in the air are filtered, disinfected and prevented from growing, and therefore, the bacteria and viruses on the filter can be prevented from multiplying.

Reference examples, embodiments and test examples are indicated below to explain the present invention in farther detail, but naturally, the present invention is not limited to these. “%” is “% by mass” in this specification unless otherwise noted.

### REFERENCE EXAMPLE 1

### Preparation of Sasa Extract

Dried leaves of Kumazasa collected in Teshio Mountains in Hokkaido Japan in September were introduced into a pressurized hot water extraction tank, treated at 125° C. for 10 minutes in the tank, the hot water was cooled down to about 80° C. by the action of a cooling water and then the resulting extract was separated from the moisture-containing solid content using a screw-press in such a manner that the moisture content of the latter was controlled to a level of about 50% by mass. Then the solid contents having a moisture content of about 50% by mass were introduced into an autoclave and heat-treated under pressure at 180° C. for 10 minutes using saturated steam. The moisture-containing solid contents thus treated were again introduced into a pressurized hot water-extraction tank and treated at 110° C. for 5 minutes to thus obtain an extract. The first and second extracts were combined, filtered through a diatomaceous earth layer, the resulting filtrate was concentrated under reduced pressure until the solid content thereof was increased to 50% by mass and the concentrate thus prepared was subjected to flow sterilization treatment at a temperature ranging from 110 to 130° C. to yield a Sasa extract.

The sulfur content of the Sasa extract thus prepared was 3850 μg/ml (7.7 mg/gram of solid content).

### REFERENCE EXAMPLE 2

### Sasa Extract Containing Organic Acids

Sasa extract containing organic acid was produced by adding 0.5 g, 1 g, 1.5 g or 2 g of malic acid per 100 g of the Sasa extract (solid content 50% by mass) produced in Reference Example 1.

### EMBODIMENT 1

### Production of Chewing Gum

Two to fifteen grams of the Sasa extract (solid content 50% by mass) produced in Reference Example 1 was added 100 g gum base heated to 60° C. for 30 minutes in a constant temperature bath, kneaded for 2 minutes, and reheated to 60° C. for 10 minutes. The kneaded mixture thus obtained was left to cool, and then was rolled to produce stick chewing gum with a thickness of approximately 1 mm (as well as chewing gum in a variety of shapes including balls).

### EMBODIMENT 2

### Production of Pills

A compound of the following composition (units in % by mass) was uniformly mixed in a kneader, and then was formed into pills in a granulator.

| | |
|---|---|
| Sasa extract of Reference Example 1 | 20 |
| (solid content 50% by mass) | |
| Gambir | 10 |
| Licorice | 15 |
| Cassia | 15 |
| Fennel | 5 |
| Mint | 5 |
| Thyme | 5 |
| Ginger | 5 |
| Clove | 5 |
| Hydrangea (Amacha) | 5 |
| Cornstarch | 8 |
| Magnesium oxide | 2 |
| Total | 100 |

### EMBODIMENT 3

### Protective Gauze

A solution of Sasa extract (solid content 50% by mass) produced in Reference Example 1 diluted 10 times was sprayed on silk gauze, and was dried for 1 hour at 80° C. to produce protective gauze containing 8.8% Sasa extract solid content by mass.

### EMBODIMENT 4

### Protective Gauze

Silk gauze was immersed in a 6-fold aqueous dilution solution of Sasa extract (solid content 50% by mass) produced in Reference Example 2 for one hour at 80° C., and was dried for one hour at 80° C. to produce protective gauze containing 8% Sasa extract solid content by mass.

### EMBODIMENT 5

### Protective Masks

Protective masks were produced by inserting the protective gauzes of Embodiments 3 and 4 on the inside of masks comprising multiple layers of gauze.

### EMBODIMENT 6

### Air Cleaner

A filter used in an ordinary air cleaner was immersed in a 6-fold aqueous dilution solution of Sasa extract (solid content 50% by mass) produced in Reference Example 2 for one hour at 80° C., and was dried for one hour at 80° C. to produce a filter containing 8% Sasa extract solid content by mass. This was installed in an air cleaner.

### TEST EXAMPLE 1

### Antibiotic Tests

The protective gauze produced in Embodiment 3 (containing Sasa extract solid content 8.8% by mass) and silk gauze not containing Sasa extract (control gauze) were used.

One milliliter of the following bacterial solutions (bacteria count approximately 5.0×10⁴ bacteria/mL or approximately 5.0×10⁵ bacteria/mL) were dripped on the respective gauzes (5×7 cm), and were left to stand and cultured for 12 hours at 37° C. in an incubator. After culturing was complete, the gauze was thoroughly rinsed with 10 mL of liquid culture medium to produce the test solution. A spiral system (NASA system, USA) was used to coat this on a brain heart agar plate culture medium. The plate was left to stand and cultured for 18 hours at 37° C. in an incubator. The colonies on the brain heart agar plate were counted, and the results are indicted in Table 1 and Table 2.

TABLE 1

| | Number of | Bacteria count after storage | |
|---|---|---|---|
| | bacteria | Gauze of the | Control |
| Bacteria | inoculated | present invention | gauze |
| Staphylococcus aureus | 5.0 × 10⁴ | 2.0 × 10(<10²) | >10⁶ |
| MRSA | 5.1 × 10⁴ | 1.0 × 10(<10²) | >10⁶ |
| Pseudomonas aeruginosa | 5.6 × 10⁴ | 5.0 × 10(<10²) | >10⁶ |
| Escherichia coli | 5.2 × 10⁴ | 8.0 × 10(<10²) | >10⁶ |
| Bacillus subtilis | 5.4 × 10⁴ | 9.0 × 10(<10²) | >10⁶ |

TABLE 2

| | Number of | Bacteria count after storage | |
|---|---|---|---|
| | bacteria | Gauze of the | Control |
| Bacteria | inoculated | present invention | gauze |
| Staphylococcus aureus | 5.4 × 10⁵ | 5.0 × 10(<10²) | >10⁷ |
| MRSA | 5.5 × 10⁵ | 6.0 × 10(<10²) | >10⁷ |
| Pseudomonas aeruginosa | 5.6 × 10⁵ | 9.0 × 10(<10²) | >10⁷ |
| Escherichia coli | 5.2 × 10⁵ | 9.8 × 10² | >10⁷ |
| Bacillus subtilis | 5.4 × 10⁵ | 8.2 × 10² | >10⁷ |

The numbers in parenthesis in Tables 1 and 2 indicate the bacteria count by general display.

In contrast to the decrease of bacteria count of the gauze containing Sasa extract of the present invention to approximately 1 in 1000 to 1 in 10000 after 12 hours of culturing, the bacteria count of the gauze not containing Sasa extract increased 100 times or more. This indicates that the gauze containing Sasa extract of the present invention has superior antibacterial characteristics, and specifically has notable antibacterial characteristics in relation to resistant bacteria MRSA on which antibiotics have no effect.

### TEST EXAMPLE 2

### Growth Suppression Tests on Anthrax Bacteria

1. Test method

Strain used: Spore solution of anthrax 34-F2 (toxogenic, encapsulated asporogenous weakened strain). This was adjusted to 34,000 bacteria per 0.1 mL.

Test solution: Solution of Sasa extract solid content 50% by mass

Investigation method: The aforementioned test solution was diluted with sterile distilled water, and adjusted to solutions of 25%, 12.5%, 6.25%, 3.2%, 1.6%, 0.8%, and 0.4%, and investigations were conducted in the following two ways. Sterile distilled water was used as the control.

1) After mixing 0.1 mL of anthrax 34-F2 spore solution in 1 mL each of the 10 types of 50 to 0% test solutions prepared above and leaving to stand for 24 hours at 37° C., 0.1 mL of this was smeared on brain heart infusion (BHI) agar medium, and after culturing for 24 hours at 37° C., colony growth was observed. The increase or decrease of bacteria count in sterile distilled water was also observed at this time.
2) A two-fold concentration of BHI agar was added to 10 mL each of the 10 types of 50 to 0% test solutions produced above to prepare agar plates. 0.1 mL of anthrax 34-F2 spore solution was smeared on the respective media, and after culturing for 24 hours at 37° C., the colony growth was observed.

### 2. Results

The results are indicated in Table 3 below.

TABLE 3
Sasa extract solid component concentration (%)

| 25 | 12.5 | 6.25 | 3.2 | 1.6 | 0.8 | 0.4 | 0.2 | 0.1 | 0 |
|---|---|---|---|---|---|---|---|---|---|
| − | − | − | − | − | − | +/− | + | + | + |

+: Colonies grow with no difference from the control
−: No colonies observed
+/−: Colony formation is observed, but there are fewer than with the control.

### 3. Conclusions

It was not confirmed that the tested Sasa extract has a power to sterilize anthrax spores, but Sasa extract did strongly suppress the growth of anthrax bacteria. As a result, the skin protective composition of the present invention containing Sasa extract can suppress the growth of anthrax bacteria, and consequently can prevent infection by anthrax bacteria.

### TEST EXAMPLE 3

### Test of Antiviral Effect on the Influenza Virus

1. Test method

Herpes simplex virus (HSV) HF strain, herpes simplex virus (HSV) UW strain, influenza virus (Inf.) A/PR/8 strain, and influenza virus (Inf.) A/FM/1/47 strain were used. Vero cells (derived from African green monkey kidney) were used as the culture cells for HSV, and MDCK cells (derived from dog liver) were used for the Inf.

After mixing 10 μL of viral solution with 1 mL of Sasa extract produced in Reference Example 1 diluted with sterile distilled water, the established cell line cultured at room temperature (23±3° C.) in a 24-well plate was inoculated with 10 μL of viral solution. This plate was cultured in a CO₂ incubator at 37° C., and the antiviral effect was judged by the extent of cytopathic effect (CPE) based on the following criteria. Distilled water (DW) was used as the control.

NT: not tested; 4+ CPE on entire surface of well; 3+ CPE on 75% or more of the well; 2+: CPE on 50 to 75% of the well; +: CPE on 25 to 50% of the well; ±: CPE on 25% or less of the well; −: CPE not observed.

For electron microscope samples, the virus was allowed to infect the culture cells for 20 hours; the cells were treated with dilute Sasa extract, and then were fixed and observed following standard methods.

### 2. Results

The antiviral effects on herpes simplex virus (HSV) HF strain, herpes simplex Virus (HSV) UW strain, influenza virus (Inf.) A/PR/8 strain, and influenza virus (Inf.) A/FM/1/47 strain are indicated in Tables 4 to 7.

TABLE 4
HSV HF strain

| | Sasa extract solid component concentration (%) | | | | | | |
|---|---|---|---|---|---|---|---|
| | 10 | 8 | 5 | 3 | 2 | 1 | Distilled water |
| 1 minute | 3+ | 4+ | 4+ | 4+ | 4+ | 4+ | 4+ |
| 5 minutes | — | ± | 4+ | 4+ | 4+ | 4+ | 4+ |
| 10 minutes | — | — | — | 2+ | 4+ | 4+ | 4+ |
| 15 minutes | — | — | — | — | ± | 2+ | 4+ |
| 30 minutes | — | — | — | — | — | — | 4+ |
| 60 minutes | — | — | — | — | — | — | 4+ |

The virus was deactivated in 5 minutes processing at a concentration of 10%, and was deactivated in 1 hour of processing even at 1% concentration.

TABLE 5
HSV UW strain

| | Sasa extract solid component concentration (%) | | | | | | |
|---|---|---|---|---|---|---|---|
| | 10 | 8 | 5 | 3 | 2 | 1 | Distilled water |
| 1 minute | — | ± | 2+ | 2+ | 3+ | 4+ | 4+ |
| 5 minutes | — | — | 1+ | ± | 2+ | 3+ | 4+ |
| 10 minutes | — | — | — | — | ± | ± | 4+ |
| 15 minutes | — | — | — | — | — | — | 4+ |
| 30 minutes | — | — | — | — | — | — | 4+ |
| 60 minutes | — | — | — | — | — | — | 4+ |

The virus was deactivated in 1 minute processing at a concentration of 10%, and was deactivated in 15 minutes of processing even at 1% concentration.

TABLE 6
Inf. A/PR/8 strain

| | Sasa extract solid component concentration (%) | | | | | |
|---|---|---|---|---|---|---|
| | 5 | 2 | 1 | 0.5 | 0.1 | Distilled water |
| 1 minute | 4+ | 4+ | 4+ | 4+ | 4+ | 4+ |
| 5 minutes | 4+ | 4+ | 4+ | 4+ | 4+ | 4+ |
| 10 minutes | 2+ | 2+ | 2+ | 2+ | 4+ | 4+ |
| 15 minutes | 2+ | 4+ | 2+ | 2+ | 4+ | 4+ |
| 30 minutes | — | — | — | — | 2+ | 4+ |
| 60 minutes | — | — | — | — | 2+ | 4+ |

The virus was deactivated in 30 minutes processing at a concentration of 0.5% or greater.

TABLE 7
Inf. A/FM/1/47 strain

| | Sasa extract solid component concentration (%) | | | | | |
|---|---|---|---|---|---|---|
| | 5 | 2 | 1 | 0.5 | 0.1 | Distilled water |
| 1 minute | 4+ | 4+ | 4+ | 4+ | 4+ | 4+ |
| 5 minutes | 4+ | 4+ | 4+ | 4+ | 4+ | 4+ |
| 10 minutes | 4+ | 4+ | 2+ | + | 2+ | 4+ |
| 15 minutes | 4+ | 4+ | — | + | 2+ | 4+ |
| 30 minutes | 4+ | 4+ | + | — | ± | 4+ |
| 60 minutes | 2+ | 2+ | — | — | — | 4+ |

The virus was deactivated in 30 minutes processing at a concentration of 0.5% or greater.

### 3. Conclusions

These results demonstrate that the anti-viral effect of Sasa extract on the influenza virus differs from that of existing preparations, but the mechanism of action is unclear.

What is claimed is:

1. A method, comprising:
suppressing the growth of a microorganism selected from the group consisting of anthrax bacteria and a virus selected from the group consisting of herpes simplex virus and influenza virus by:
providing an object containing or impregnated with a composition comprising Sasa extract, and
bringing the microorganism into contact with the composition.

2. The method of claim 1, wherein the microorganism is anthrax bacteria.

3. The method of claim 2, wherein the composition further comprises an organic acid.

4. The method of claim 3, wherein the organic acid is malic acid.

5. The method of claim 2, wherein the composition is in the form of a protective cloth.

6. The method of claim 2, wherein the composition is in the form of gauze, mask, eye patch, menstrual band, menstrual napkin, medical dressing, toilet paper, gauze for treating hemorrhoids, toilet seat sheet, shoe insert, ear plug, glove, hat, white gown, sheet, futon cover, pillow case, curtain, wall paper, carpet, or surgical suture thread.

7. The method of claim 2, wherein the composition is in the form of gauze.

8. The method of claim 2, wherein the composition is in the form of mask.

9. The method of claim 1, wherein the microorganism is a virus selected from the group consisting of herpes simplex virus and influenza virus.

10. The method of claim 9, wherein the composition further comprises an organic acid.

11. The method of claim 10, wherein the organic acid is malic acid.

12. The method of claim 9, wherein the composition is in the form of a protective cloth.

13. The method of claim 9, wherein the composition is in the form of gauze, mask, eye patch, menstrual band, menstrual napkin, medical dressing, toilet paper, gauze for treating hemorrhoids, toilet seat sheet, shoe insert, ear plug, glove, hat, white gown, sheet, futon cover, pillow case, curtain, wall paper, carpet, or surgical suture thread.

14. The method of claim 9, wherein the composition is in the form of gauze.

15. The method of claim 9, wherein the composition is in the form of mask.

16. The method of claim 9, wherein the composition is in the form of an oral composition.

17. The method of claim 9, wherein the composition is in the form of a confection, breads, noodles, beverages, tablets, pills, mouthwash, gargle, toothpaste, skin adhesive film, or spray agent for treating throat inflammation.

18. The method of claim 9, wherein the confection is in the form of a gummi, jelly, troche, candy, chewing gum, jam, sherbet, cream, drop, sponge cake, cookie, chocolate, or rice cracker.
